(19) United States
(12) Patent Application Publication
(10) Pub. No.: US 2022/0323257 A1
(43) Pub. Date: Oct. 13, 2022

(54) WELDING HELMET CAMERA SYSTEM

(71) Applicant: Saul Quijada, Phoenix, AZ (US)

(72) Inventor: Saul Quijada, Phoenix, AZ (US)

(21) Appl. No.: 17/228,709

(22) Filed: Apr. 13, 2021

(51) Int. Cl. A61F 9/06; G02B 27/01

(52) U.S. Cl. CPC A61F 9/06; G02B 27/0172

(57) ABSTRACT

A welding helmet camera system; the welding helmet camera system includes a welding helmet for covering a substantial portion of a head of a user-wearer during use and providing enhanced vision and protection for a user-wearer's eyes and face from flashes, sparks, extreme brightness and flying debris. The welding helmet includes a first-camera, a second-camera, a first-display screen, and a second-display screen. The first-display screen is in communication with the first-camera and is configured to broadcast digital video generated by the first-camera during a welding operation. The second-display screen positioned on the interior surface of the body and is in communication with the second-camera and is configured to display another digital video generated by the second-camera during the welding operation.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of welding helmets of existing art and more specifically relates to welding helmets having cameras.

### RELATED ART

Welding in general is a fabrication process that joins pieces of materials, for example, metals, together in a permanent manner by causing fusion of the materials. Such fusion of adjacent pieces of metal requires enough energy to melt the metal and bond the pieces before cooling. Arc welding is a typical welding method in which the energy necessary to melt the metal is provided by a high voltage electric arc between at least one of the metal pieces and a metal electrode that slowly melts away at the point where the electric arc emanates from the electrode to create a puddle of electrode metal which fuses together with the adjacent metal pieces. The electric arcs that create the heat necessary to melt the metals in arc welding also create very intense, high energy radiation emissions, e.g., extremely bright visible light, ultraviolet, and infrared radiation. Such radiation is so intense that a person cannot look at an ongoing arc welding process without an extremely high risk of flash burns in which high intensity ultraviolet radiation causes inflammation of the cornea and can burn the retina of the person's eyes. Therefore, goggles or welding helmets with dark, ultraviolet filtering face plates have to be worn by welders to prevent these kinds of eye damage. The problem is the polarized glass eye shield of currently available welding helmets often gets scratched and discolored over time. These scratches and the discoloration can distort the user's vision and impair their welding performance. So, while these ordinary helmets protect the user's eyes and face from flashes, sparks, extreme brightness and flying debris a suitable alternative is desired.

U.S. Pat. No. 10,701,270 to Thomas J. LaFlech relates to a camera display welder's helmet. The described camera display welder's helmet includes a light blocking welding helmet having a helmet body with a shell having a head aperture produces an image from at least one (1) exterior camera within the helmet body. The helmet body includes an interior cavity containing an adjustable headband and an internal power source for powering the viewing screen and camera. The camera is mounted on the helmet body. Electrical circuitry is operatively connected to the power source, to the viewing screen, and to the camera such that the electrical circuitry causes the viewing screen to display the image produced by the camera. The image can be transmitted to a remote receiving unit where a weld can be monitored. Multiple cameras may be used and a control switch then selects which camera that will produce an image.

### SUMMARY OF THE INVENTION

In view of the foregoing disadvantages inherent in the known welding helmets art, the present disclosure provides a novel welding helmet camera system. The general purpose of the present disclosure, which will be described subsequently in greater detail, is to provide a welding helmet camera system that provides improved vision, increased protection, performance and remote inspection capabilities.

A welding helmet camera system is disclosed herein. The welding helmet camera system includes a welding helmet comprising a body configured to enclose a substantial portion of a head of a user-wearer during use and provide protection for a user-wearer's eyes and face from flashes, sparks, extreme brightness and flying debris. The welding helmet further comprises a first-camera, a second-camera, a first-display screen, a second-display screen, a rechargeable power source configured to provide power to the welding helmet, a port, and a control panel. The first-display screen is positioned on an interior surface of the body and is in communication with the first-camera. The first-display screen is configured to broadcast digital video generated by the first-camera during a welding operation. The second-display screen is also positioned on the interior surface of the body and is in communication with the second-camera. The second-display screen is configured to display digital video generated by the second-camera during the welding operation.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a welding helmet camera system, constructed and operative according to the teachings of the present disclosure.

FIG. 1 is a perspective view of the welding helmet camera system during an ‘in-use’ condition, according to an embodiment of the disclosure.

FIG. 2 is a perspective view of the welding helmet camera system of FIG. 1, according to an embodiment of the present disclosure.

FIG. 3 is a rear view of the welding helmet camera system of FIG. 1, according to an embodiment of the present disclosure.

FIG. 4 is a side perspective view of the welding helmet camera system of FIG. 1, according to an embodiment of the present disclosure.

FIG. 5 is a perspective view of the welding helmet camera system of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a welding helmet with cameras and more particularly to a welding helmet camera system as used to improve welder vision and protection during a welding operation.

Generally, the welding helmet camera system provides a welding helmet having at least a first-camera and a second-camera mounted to an exterior front portion of the welding helmet and at least one high-definition screen positioned within the welding helmet which is configured to display footage captured from the first-camera and the second-camera. The screen may be slightly curved to give the user-wearer a full range of vision. The video cameras and monitors replace the lens shade used by conventional welding helmets. The welding helmet camera system allows users to magnify and zoom in on work. Buttons for control of the cameras and zooming of their lenses, as well as buttons for control of the video monitors display, are featured on the welding helmet. By allowing magnification and zooming, the welding helmet camera system permits users to have sharper visual focus. The cameras automatically adjust to protect the user-wearer's eyes while welding. Additionally, the first-camera and the second-camera may auto adjust to accommodate for different brightness's of various welding environments. Clear, heat-resistant caps may be provided. The heat-resistant caps may be removably clipped over the camera lenses of the first-camera and the second-camera to protect them from sparks and flying debris.

Furthermore, buttons for zoom, brightness, and other functions are provided on the welding helmet to allow for manual adjustment. The first-camera and the second-camera may record and/or stream video during or after a weld allowing a secondary party to inspect weld performance. To accommodate this functionality a CPU, transceiver, Bluetooth® and Wi-Fi technology may be utilized. The welding helmet features a rechargeable battery and charging cord. In another embodiment, the helmet features exterior photovoltaic cells to allow for solar charging. The welding helmet camera system may include one, two or more monitors. The welding helmet camera system may include one, two or more video cameras. The welding helmet camera system may include a fan for cooling the interior of the welding hood.

In a preferred embodiment, the welding helmet of the welding helmet camera system is made in the same design and with similar materials as a standard welding helmet, but with a closed face plane that has no standard lens or lens shade. Featured in strategic locations within the front face plane of the helmet, and with lenses that are protected by replaceable, translucent, heat-resistant caps, are two video cameras. The video cameras are of high-definition standards. The aforementioned video cameras connect to and broadcast upon two video monitors, strategically placed within the interior of the helmet. The monitors are slightly curved. The video cameras include self-adjustment capabilities that respond to brightness of the received video. The cameras also include zoom features as well as many other camera related features.

Buttons for manual control of the cameras and monitors, such as zoom and brightness, are featured on an external side plane of the helmet. A memory chip, located within the interior lining of the helmet, connects by wire to the video cameras for recording of their broadcast. Additionally, a central processing unit with transceiver, Bluetooth® and Wi-Fi compatibility are featured to allow broadcast of the cameras video in real time. A rechargeable battery connects by wire to the cameras, monitors, and memory chip. The welding helmet camera system may or may not include a memory chip for video recording. An exterior port accepts an included power cord for recharging of this battery.

In one embodiment, the welding helmet features at least one exterior illumination member to provide illumination. In another embodiment, the exterior of the helmet features photovoltaic cells in order to charge the battery when the welding helmet is used outdoors during daylight hours. The welding helmet camera system may include a secondary camera accessory hosted upon an extendable and flexible arm. The accessory may be a standalone item, or it may attach to the actual welding helmet. The flexible arm may be adjusted and bent such that “hard-to-see” spots can become clearly visible upon the monitors of the welding helmet. In other embodiments, the welding helmet camera system may be configured for use in underwater welding. The welding helmet camera system may include an internal microphone, an internal audio speaker, or both microphone and speaker to accept communication from and/or to others who are not in the immediate area but who are observing user work.

The welding helmet camera system can be made in various sizes and shapes, and may be produced of various materials of adequate durability and heat tolerance. The monitors of the welding helmet camera system may be of various pixel resolutions and of various radii in curvature. The lenses of the welding helmet camera system may be of various zoom magnification limits. The welding helmet camera system may be made in a simplified version with a clear shielded receptacle for a smart phone and the camera, zoom and brightness control all integrated in a software app on that phone. The welding helmet camera system may include a simulcast function in its cameras, which could transmit its reception to other devices, and to allow others to view work of the wearer from the same visual perspective as the wearer during use. A gauge that displays the power status of the battery may be included, and the gauge may appear on the monitors of the welding helmet camera system or may be in any strategic location.

A method of using the welding helmet camera system is as follows: After ensuring its batteries to be of charged status, a user may simply turn on the welding helmet camera system and don the device upon his or her head as a helmet. The first-camera and the second-camera of the device will then broadcast images directly in front of the wearer to the first-display screen and the second-display screen. The user may then conduct welding work, and can adjust a lens of the first-camera and the second-camera to magnify a focal visual point when desired. At no time will vision of the user be distorted or weakened, as ordinarily occurs with standard welding masks with lens shades that become aged, discolored, or damaged. The video broadcast will also be captured on the memory chip of the welding helmet from which previous work can be reviewed. Additionally, a welder's work can be streamed in real time.

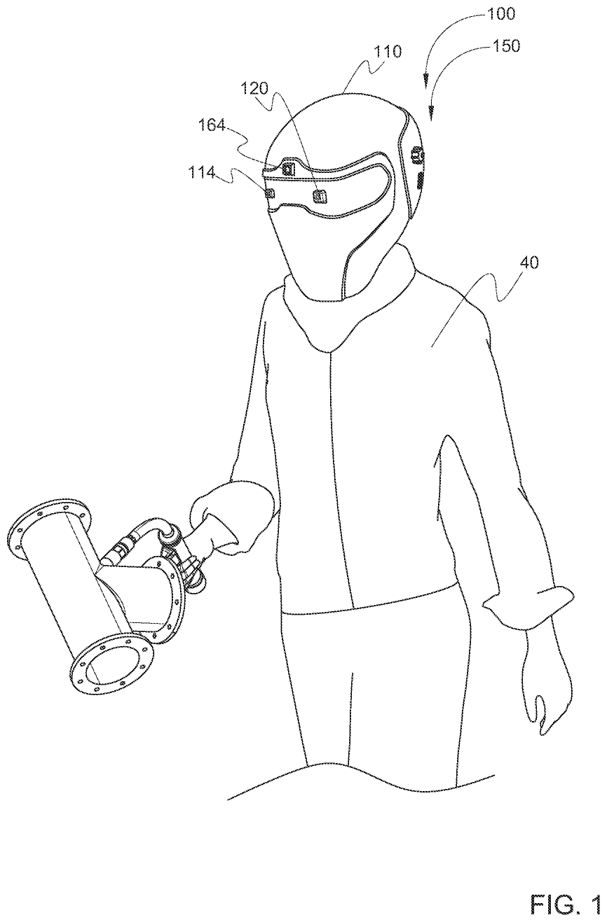

Referring now more specifically to the drawings by numerals of reference, there is shown in FIGS. 1-5, various views of a welding helmet camera system 100.

FIG. 1 shows a welding helmet camera system 100 during an ‘in-use’ condition 150, according to an embodiment of the present disclosure. Here, the welding helmet camera system 100 may be beneficial for use by a user-wearer 40 to provide protection and improved vision. As illustrated, the welding helmet camera system 100 may include a welding helmet 110 including a body 112 configured to enclose a substantial portion of a head of a user-wearer 40 during use and provide protection for a user-wearer's eyes and face from flashes, sparks, extreme brightness and flying debris. The welding helmet 110 further comprises a first-camera 114, a second-camera 120, a first-display screen 124, a second-display screen 130, a rechargeable power source 134 configured to provide power to the welding helmet 110, a port 136, and a control panel 140. The welding helmet 110 includes wireless connect-ability features. The welding helmet 110 provides a user-wearer 40 with improved vision during use.

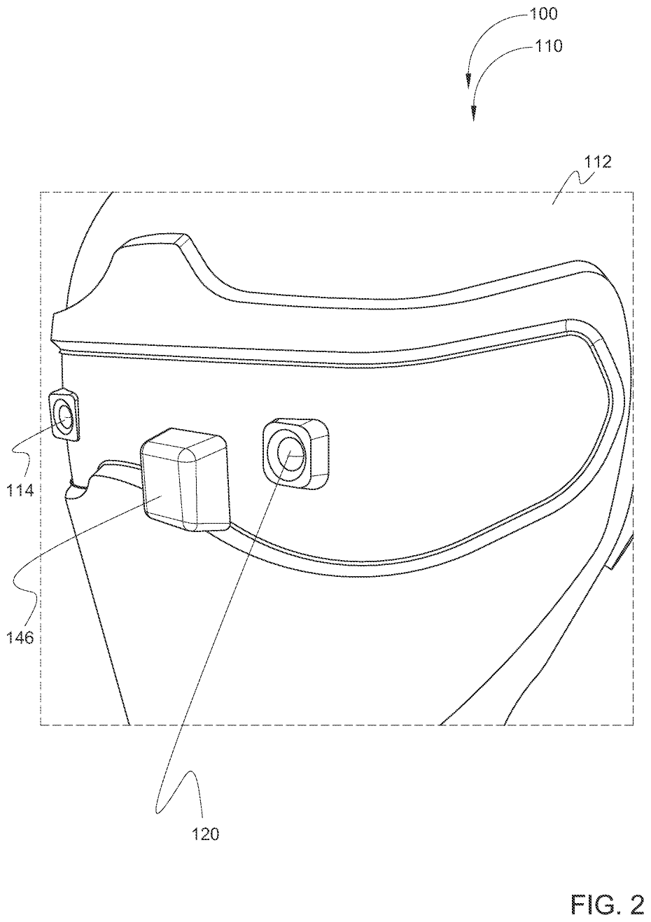

FIG. 2 shows the welding helmet camera system 100 of FIG. 1, according to an embodiment of the present disclosure. As above, the welding helmet camera system 100 may include the welding helmet 110 having the first-camera 114 and the second-camera 120 mounted on a frontal exterior portion of the body 112. The first-camera 114 and the second-camera 120 and are in communication with a memory chip 166, a central processing unit 168, and a transceiver 170. The first-camera 114 and the second-camera 120 each comprise a high-definition video camera. The first-camera 114 and the second-camera 120 are configured to automatically adjust a displayed-brightness according to a brightness of a welding environment during the welding operation in order to protect the eyes of the user-wearer 40 during use. By adjusting in brightness, the welding helmet camera system 100 protects the vision of its wearer from damage caused by bright lighting displayed by a welding iron. The welding helmet camera system 100 ensures ideal visibility for the user-wearer 40 in all environments of use, such as but not limited to indoor and outdoor environments, and daytime and nighttime use. As shown, heat resistant lens caps 146 are included to cover the first-camera 114 and the second-camera 120 and provide protection for the first-camera 114 and the second-camera 120. The heat resistant lens caps 146 are transparent and shatter resistant. The heat resistant lens caps 146 are removable and replaceable as needed.

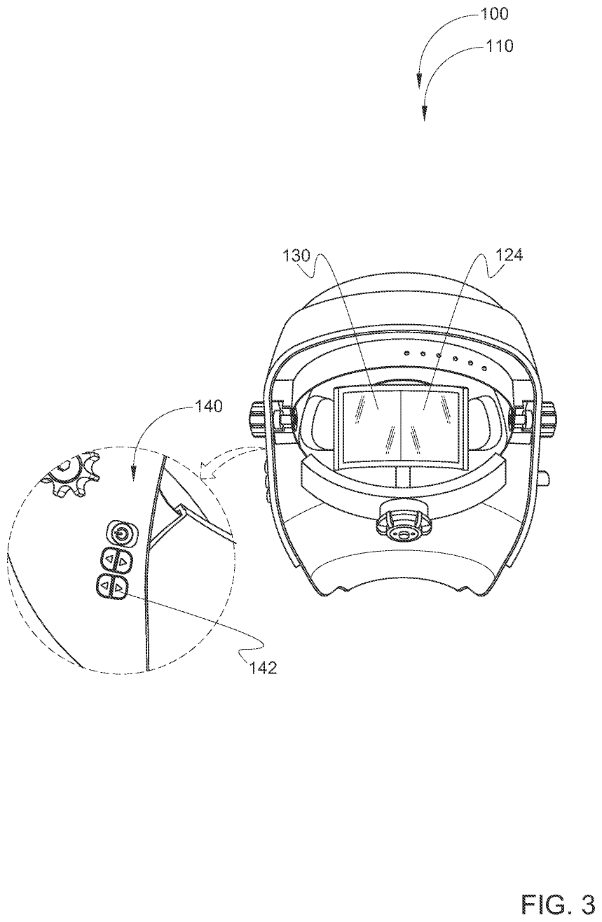

FIG. 3 is a rear view of the welding helmet camera system 100 of FIG. 1, according to an embodiment of the present disclosure. The body 112 further includes the first-display screen 124 and the second-display screen 130 positioned on an interior surface, as further illustrated in FIG. 3. The first-display screen 124 and the second-display screen 130 each comprise a high-definition display screen. The first-display screen 124 is in communication with the first-camera 114 and is configured to broadcast digital video generated by the first-camera 114 during a welding operation. The second-display screen 130 positioned on the interior surface of the body 112 and is in communication with the second-camera 120 and is configured to display another digital video generated by the second-camera 120 during the welding operation.

In a preferred embodiment, the first-display screen 124 and the second-display screen 130 each comprise a curved profile for increasing a field of vision for a user-wearer 40 during use. The curved monitors of the welding helmet camera system 100 eliminate the distortion in vision that ordinary lens shades can produce. The first-display screen 124 and the second-display screen 130 are in alignment with the eyes of the user-wearer 40 during wear. The digital video generated by the second-camera 120 and the first-camera 114 is displayed on the first-display screen 124 and the second-display screen 130 in real time. In other scenarios, the digital video may be streamed to a remote viewer. Additionally, the digital video may be recorded and stored for later viewing. The memory chip 166 stores digital video generated by the second-camera 120 and the first-camera 114 for viewing after the welding operation is complete. The welding helmet camera system 100 may include an internal microphone for audio recording, savable on the memory chip 166, which would coincide with and broadcast with the digital video. Additionally, the welding helmet camera system 100 may include an internal microphone connected to an external audio speaker to allow user communication with others in the immediate area.

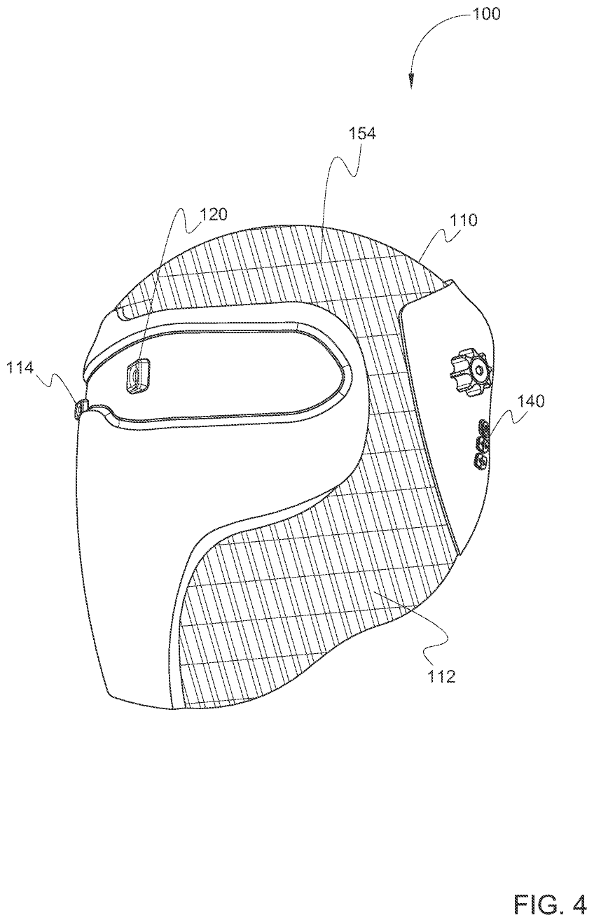

FIG. 4 is a side perspective view of the welding helmet camera system 100 of FIG. 1, according to an embodiment of the present disclosure. The welding helmet camera system 100 includes the welding helmet 110 comprising the first-camera 114, the second-camera 120, the first-display screen 124, and the second-display screen 130 working in functional combination to provide improved vision and protection for a user-wearer 40 while performing welding operations. As shown, the welding helmet 110 further comprises the control panel 140 includes a plurality of buttons 142 allowing for manual user control of at least one function of the welding helmet 110. The at least one function of the control panel 140 of the welding helmet 110 includes power control, brightness control, and camera zoom control. The control panel 140 may be positioned on a side exterior portion of the body 112 of the welding helmet 110. In certain embodiments, the welding helmet camera system 100 may include a remote-control device for its operation.

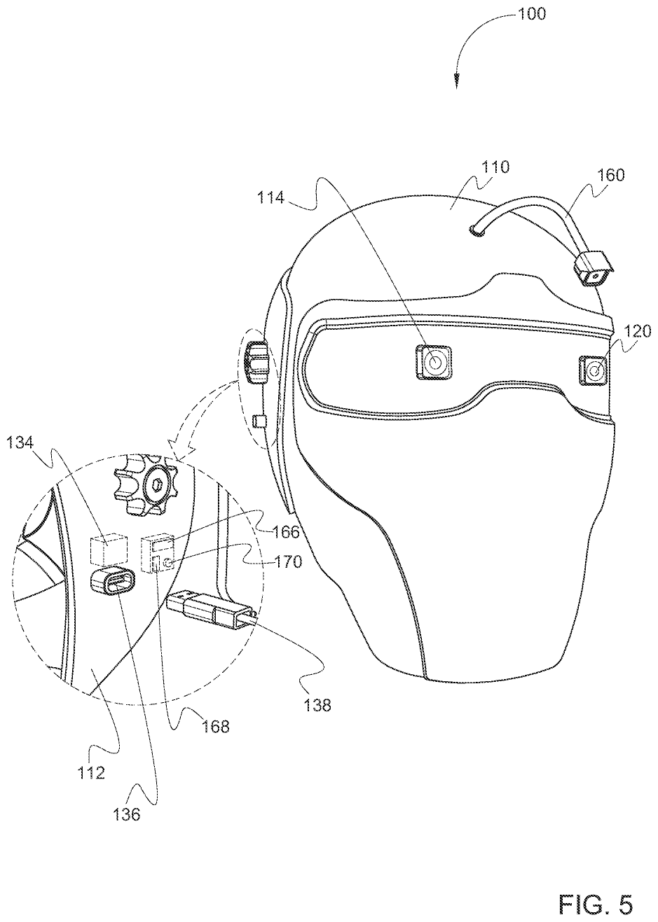

FIG. 5 is a perspective view of the welding helmet camera system 100 of FIG. 1, according to an embodiment of the present disclosure. As illustrated, the welding helmet 110 includes the port 136 configured to receive a charger 138 for charging the rechargeable power source 134. In one embodiment, the power source is a rechargeable battery. In other embodiments, the welding helmet 110 further comprises solar panels 154, as shown in FIG. 4, which are configured to solar charge the rechargeable battery. The welding helmet 110 provides increased protection, performance and remote inspection capabilities. In one embodiment the welding helmet 110 features at least one exterior illumination member 164, controlled by the user, to illuminate areas that are being welded. The welding helmet may further comprise an additional camera-accessory 160 extending from said welding helmet. The additional camera-accessory 160 may be hosted upon an extendable and flexible arm.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A welding helmet camera system, the welding helmet camera system comprising:
a welding helmet including,
a body configured to enclose a substantial portion of a head of a user-wearer during use,
a first-camera and a second-camera mounted on a frontal exterior portion of said body and in communication with a memory chip, a central processing unit and a transceiver,
heat resistant lens caps configured to cover lens on said first-camera and said second-camera,
a first-display screen positioned on an interior surface of said body and in communication with said first-camera and being configured to broadcast digital video generated by said first-camera during a welding operation, and
a second-display screen positioned on said interior surface of said body and in communication with said second-camera and being configured to display another digital video generated by said second-camera during said welding operation,
a rechargeable power source configured to provide power to said welding helmet,
a port configured to receive a charger for charging said rechargeable power source, and
a control panel.

2. The welding helmet camera system of claim 1, wherein said first-camera and said second-camera each comprise a high-definition video camera.

3. The welding helmet camera system of claim 1, wherein said first-camera and said second-camera are configured to automatically adjust a displayed-brightness according to a brightness of a welding environment during said welding operation in order to protect eyes of said user-wearer during use.

4. The welding helmet camera system of claim 1, wherein said first-display screen and said second-display screen each comprise a curved profile.

5. The welding helmet camera system of claim 4, wherein said first-display screen and said second-display screen are in alignment with the eyes of said user-wearer during wear.

6. The welding helmet camera system of claim 1, wherein said first-display screen and said second-display screen each comprise a high-definition display screen.

7. The welding helmet camera system of claim 1, wherein said welding helmet further comprises at least one exterior illumination member.

8. The welding helmet camera system of claim 1, wherein said control panel includes a plurality of buttons allowing for manual user control of at least one function of said welding helmet.

9. The welding helmet camera system of claim 1, wherein said at least one function of said control panel of said welding helmet includes power control, brightness control, and camera zoom control.

10. The welding helmet camera system of claim 1, wherein said digital video generated by said second-camera and said first-camera is displayed on said first-display screen and said second-display screen in real time.

11. The welding helmet camera system of claim 1, wherein said memory chip stores digital video generated by said second-camera and said first-camera for viewing after said welding operation is complete.

12. The welding helmet camera system of claim 11, wherein said control panel is positioned on a side exterior portion of said body of said welding helmet.

13. The welding helmet camera system of claim 1, wherein said welding helmet is able to connect to an internet.

14. The welding helmet camera system of claim 1, wherein said heat resistant lens caps are transparent.

15. The welding helmet camera system of claim 14, wherein said heat resistant lens caps are removable and replaceable.

16. The welding helmet camera system of claim 15, wherein said heat resistant lens caps are shatter resistant.

17. The welding helmet camera system of claim 1, wherein said rechargeable power source comprises a rechargeable battery.

18. The welding helmet camera system of claim 17, wherein said welding helmet further comprises solar panels configured to solar charge said rechargeable battery.

19. The welding helmet camera system of claim 1, wherein said welding helmet further comprises an additional camera-accessory extending from said welding helmet.

20. A welding helmet camera system, the welding helmet camera system comprising:
a welding helmet including,
a body configured to enclose a substantial portion of a head of a user-wearer during use,
a first-camera and a second-camera mounted on a frontal exterior portion of said body and in communication with a memory chip, a central processing unit and a transceiver,
heat resistant lens caps configured to cover lens on said first-camera and said second-camera,
a first-display screen positioned on an interior surface of said body and in communication with said first-camera and being configured to broadcast digital video generated by said first-camera during a welding operation, and
a second-display screen positioned on said interior surface of said body and in communication with said second-camera and being configured to display another digital video generated by said second-camera during said welding operation,
a rechargeable power source configured to provide power to said welding helmet,
a port configured to receive a charger for charging said rechargeable power source, and
a control panel,
wherein said first-camera and said second-camera each comprise a high-definition video camera;
wherein said first-camera and said second-camera are configured to automatically adjust a displayed-brightness according to a brightness of a welding environment during said welding operation in order to protect eyes of said user-wearer during use;
wherein said first-display screen and said second-display screen each comprise a curved profile;
wherein said first-display screen and said second-display screen are in alignment with the eyes of said user-wearer during wear;
wherein said first-display screen and said second-display screen each comprise a high-definition display screen;
wherein said control panel includes a plurality of buttons allowing for manual user control of at least one function of said welding helmet;
wherein said at least one function of said control panel of said welding helmet includes power control, brightness control, and camera zoom control;
wherein said memory chip stores digital video generated by said second-camera and said first-camera for viewing after said welding operation is complete;
wherein said control panel is positioned on a side exterior portion of said body of said welding helmet;
wherein said welding helmet is able to connect to an internet;
wherein said heat resistant lens caps are transparent;
wherein said heat resistant lens caps are removable and replaceable;
wherein said heat resistant lens caps are shatter resistant; and
wherein said rechargeable power source comprises a rechargeable battery.
